(19) United States
(12) Patent Application Publication
(10) Pub. No.: US 2009/0324526 A1
(43) Pub. Date: Dec. 31, 2009

(54) Compositions comprising madecassoside and/or terminoloside and an arginine and/or a salt and/or a derivative thereof for inducing and/or stimulating the growth of human keratin fibers and/or preventing loss thereof

(75) Inventors: Pascale Lazzeri-Vigouroux, Puyricard (FR); Andre Rougier, Bessancourt (FR)

Correspondence Address: BUCHANAN, INGERSOLL & ROONEY PC, POST OFFICE BOX 1404, ALEXANDRIA, VA, 22313-1404, US

(73) Assignee: L'OREAL, PARIS (FR)

(21) Appl. No.: 12/382,887

(22) Filed: Mar. 26, 2009

Related U.S. Application Data
(60) Provisional application No. 61/043,649, filed on Apr. 9, 2008.

(30) Foreign Application Priority Data
Mar. 28, 2008 (FR) 0852009

(51) Int. Cl. A61K 8/49; A61Q 7/00

(52) U.S. Cl. 424/70.1

(57) ABSTRACT

Cosmetic care compositions for human keratin fibers contain, formulated into a physiologically acceptable medium:
a) one or more compounds selected from among arginines and/or the organic or inorganic acid salts and/or derivatives thereof;
b) a compound or a mixture of compounds of formula (I) below:

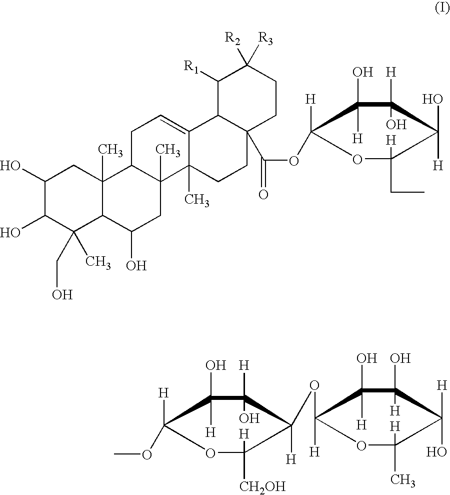

in which:
R₁═H or —CH₃; R₂═H or —CH₃; R₃═—CH₃, with the proviso that R₁ and R₂ are not simultaneously H, or an extract of Centella asiatica containing the compound or the mixture of compounds of formula (I); such compositions are useful for treating human keratin fibers and/or the scalp, for inducing and/or stimulating the growth of these fibers and/or preventing the loss and/or increasing the density thereof.

### CROSS-REFERENCE TO PRIORITY/PROVISIONAL APPLICATIONS

This application claims priority under 35 U.S.C. §119 of FR 0852009, filed Mar. 28, 2008, and of U.S. Provisional Application No. 61/043,649, Apr. 9, 2008, each hereby expressly incorporated by reference and each assigned to the assignee hereof.

### BACKGROUND OF THE INVENTION

1. Technical Field of the Invention

The present invention relates to care compositions for human keratin fibers, and in particular for reducing and/or preventing their loss and/or for inducing and/or stimulating their growth and/or increasing their density, comprising, formulated into a physiologically acceptable medium, at least:

a) an arginine and/or an organic or inorganic acid salt thereof and/or a derivative thereof; and

b) a compound or a mixture of compounds of formula (I) below or else an extract of Centella asiatica comprising same.

The present invention also relates to a cosmetic treatment regime or regimen for inducing and/or stimulating the growth of human keratin fibers and/or preventing their loss and/or increasing their density, and also to the use of the subject combination for inducing and/or stimulating the growth of these keratin fibers and/or preventing their loss.

The human keratin fibers to which the invention relates are in particular the hair, the eyebrows, the eyelashes, beard hair, moustache hair and pubic hair. More especially, this invention relates to human hair and/or human eyelashes.

2. Description of Background and/or Related and/or Prior Art

In humans, hair growth and hair renewal are mainly determined by the activity of the hair follicles and of their dermo-epidermal environment. Their activity is cyclic and comprises essentially three phases, namely, the anagenic phase, the catagenic phase and the telogenic phase.

The anagenic phase (active phase or growth phase), which lasts several years and during which the hair gets longer, is followed by a very short and transient catagenic phase which lasts a few weeks. During this phase, the hair undergoes a change, the follicle becomes atrophied and its dermal implantation appears higher and higher.

The terminal phase, which lasts a few months, corresponds to a resting phase, also known as telogenic phase. At the end of this resting period, the hairs fall out and a new cycle begins again.

The head of hair is thus under constant renewal and, out of the approximately 150,000 hairs that make up a head of hair, at any moment, approximately 10% of them are at rest and will therefore be replaced within a few months.

The literature suggests many reasons leading to early hair loss; in particular, this early loss occurs in genetically predisposed individuals and it affects in particular men. It is more particularly a matter of androgenetic or androgenic or alternatively androgenogenetic alopecia.

This alopecia is essentially due to a disturbance in hair renewal, resulting, in a first stage, in acceleration of the frequency of the cycles to the detriment of the quality of the hair, and then of the quantity thereof. A gradual depletion of the head of hair occurs by regression of the “terminal” hairs at the down stage. Certain areas are preferentially affected; especially the temporal or frontal lobes in men and also the upper part of the occipital lobe, whereas in women, a diffuse alopecia of the crown of the head is more noted.

Other causes may lead to considerable temporary or permanent hair loss. This may be hair loss and alteration after a pregnancy (postpartum), during states of undernourishment or of dietary imbalance or else during states of asthenia or of hormone dysfunction, as may be the case during or after the menopause. This may also be hair loss or alterations in relation to seasonal phenomena.

For many years, in particular in the cosmetics industry, compositions for eliminating or reducing the effect of alopecia, and in particular inducing or stimulating hair growth and/or the growth of certain body hairs and/or reducing or delaying hair loss, have been sought.

WO99/13717 discloses D-arginine, D,L-arginine, L-arginine and the salts thereof or the derivatives thereof as active agents for use in the cosmetic treatment of hair loss, by topical application. However, their effectiveness remains limited.

EP277455, FR2606634 and JP7010722 disclose that extracts of Centella asiatica are known to be active agents for the prevention of hair loss and/or for hair growth. In WO2005/123032, it has been proposed to use a combination of an extract of Centella asiatica, of an extract of coffee and of an antioxidant in compositions for use in the prevention of hair loss and/or in hair regrowth. However, their effectiveness remains limited.

Moreover, WO2004/062678 describes an extract of Centella asiatica rich in madecassoside and in terminoloside, and the use thereof in the regulation of inflammatory mechanisms.

### SUMMARY OF THE INVENTION

It has now surprisingly been discovered that the combination a) of an arginine and/or of an organic or inorganic acid salt thereof and/or a derivative thereof, and b) of a compound or of a mixture of compounds of formula (I), which will be defined below, or else of an extract of Centella asiatica comprising said compound(s) of formula (I), provides a substantially greater anti-hair loss effectiveness than each of the active agents used alone.

The present invention thus features care compositions for human keratin fibers, comprising, formulated into a physiologically acceptable medium:

a) one or more compounds selected from among arginines and/or the organic or inorganic acid salts thereof and/or the derivatives thereof; and

b) a compound or a mixture of compounds of formula (I) below or else an extract of Centella asiatica comprising a compound or a mixture of compounds of formula (I).

This invention also features a cosmetic regime or regimen of the combination a) of one or more compounds selected from among arginines and/or the organic or inorganic acid salts thereof and/or the derivatives thereof, and b) of a compound or a mixture of compounds of formula (I) or else of an extract of Centella asiatica comprising a compound or a mixture of compounds of formula (I), for caring for human keratin fibers, for inducing and/or stimulating the growth of said fibers and/or preventing their loss and/or increasing their density.

The expression “increasing the density of human keratin fibers, and in particular hair density” means increasing the number of keratin fibers, in particular of hairs, per cm² of skin such as the scalp. Thus, the subject compositions make it possible to maintain the head of hair in good condition and/or to combat natural hair loss, in particular in men.

In particular, the present invention features the cosmetic administration of the above combination in a hair care composition for treating alopecia of natural origin, and in particular androgenic or androchronogenetic alopecia.

The present invention also features a cosmetic treatment regime or regimen for human keratin fibers and/or for the scalp, for inducing and/or stimulating the growth of said fibers, in particular the hair and certain body hairs, such as the eyelashes, and/or preventing their loss and/or increasing their density, which comprises applying, to these fibers and/or the scalp, a cosmetic composition as defined above, maintaining said composition in contact with these keratin fibers and/or the scalp, and optionally rinsing these keratin fibers and/or the scalp.

This treatment regime or regimen has the characteristics of a cosmetic process insofar as it makes it possible to improve the aesthetics of human keratin fibers, and in particular of hair and of eyelashes, by providing with greater vigor and an improved appearance. In addition, it may be used daily for several months.

### DETAILED DESCRIPTION OF BEST MODE AND SPECIFIC/PREFERRED EMBODIMENTS OF THE INVENTION

The compositions in accordance with the invention comprise, as first active agent, an arginine and/or else a salt thereof and/or else a derivative thereof.

The various forms of arginine are, in general, D-arginine, D,L-arginine and L-arginine.

Among the organic or inorganic acid salts of arginine, exemplary are the hydrochlorides, glutamates, butyrates and glycolates.

For the purpose of the invention, the term “arginine derivatives” means arginines bearing substitutions either on the carboxyl group of said arginines or on the amino group borne by the carbon in the alpha-position with respect to the carboxyl group or, optionally, on these two groups.

Among the L-arginine derivatives, exemplary are the C₁-C₄ alkyl esters of L-arginine, the alkyl being selected, for example, from among methyl, ethyl, propyl, isopropyl, butyl and isobutyl.

Among the L-arginine derivatives, exemplary are the oligomers of L-arginine (known as polyarginine), such as those described in EP1060739 and in WO 03/072039.

Arginine oligomers which are preferred according to the invention comprise from 7 to 15 L-arginine units, for example 7, 9, 11, 13 or 15 L-arginine units.

L-arginine is more particularly preferred.

The arginine(s) and/or the salts thereof and/or the derivatives thereof in accordance with the invention will be present in the compositions in amounts preferably ranging from 0.01% to 5% of the total weight of the composition, and more preferably in an amount representing from 0.1% to 5% of the total weight of the composition, and even more preferably from 1% to 5%.

The compound(s) of formula (I) in accordance with the invention correspond(s) to the formula below:

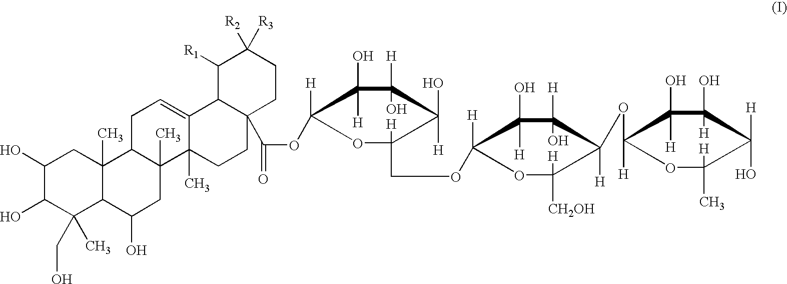

in which:

R₁═H or —CH₃; R₂═H or —CH₃; R₃═—CH₃, with the proviso that R₁ and R₂ are not being simultaneously H.

The preferred compounds of formula (I) are madecassoside (compound of formula (I) with R₁═R₃═—CH₃ and R₂═H) and terminoloside (compound of formula (I) with R₁═H and R₂═R₃═—CH₃).

These compounds of formula (I), and in particular mixtures thereof, can in particular be extracts of Centella asiatica according to the method described in WO 2004/062678.

According to one preferred embodiment of the invention, a mixture of madecassoside and of terminoloside is formulated. More preferably, the mixture of madecassoside and of terminoloside has a madecassoside content ranging from 30% to 70% by weight relative to the total weight of the mixture, and more particularly 50% by weight of madecassoside.

According to a particularly preferred embodiment of the invention, an extract of Centella asiatica comprising more than 95% by weight of the madecassoside/terminoloside mixture, relative to the weight of the extract, will be formulated.

An extract of Centella asiatica comprising more than 95% by weight of mixture of madecassoside/terminoloside (50/50% by weight) is in particular marketed under the trademark MADECASSOSIDE by BAYER. The CTFA name of this mixture is MADECASSOSIDE.

The compound or the mixture of compounds of formula (I) described above may be present in the compositions for topical application in an amount ranging, for example, from 0.001% to 20% by weight, and preferably from 0.01% to 5% by weight, relative to the total weight of the composition.

Physiologically Acceptable Medium:

The compounds used according to the invention may in particular constitute cosmetic and dermatological compositions. For such an application, they contain a physiologically acceptable medium. The term “physiologically acceptable medium” means a medium compatible with the skin, and optionally with the lips, the scalp, the eyelashes, the eyes and/or the hair.

The compositions according to the invention may be applied topically to the human scalp or human keratin fibers (to any area of skin or fibers to be treated).

These compositions may be in any of the known galenical forms suitable for the method of administration.

For topical application to the skin, including the scalp, the composition may be in the form of an aqueous, alcoholic, aqueous-alcoholic or oily solution or suspension, an emulsion or dispersion with a more or less fluid, and in particular liquid or semi-liquid, consistency, obtained by dispersion of a fatty phase in an aqueous phase (O/W) or vice versa (W/O), a solid dispersion or emulsion (O/W) or (W/O), an aqueous, aqueous-alcoholic or oily gel which is more or less fluid or solid, a loose or compacted powder to be used as it is or to be incorporated into a physiologically acceptable medium, or else microcapsules or microparticles, or vesicular dispersions of ionic and/or non-ionic type.

A composition in the form of a foam or else in the form of a spray or of an aerosol, which then comprises a pressurized propellant, is also intended.

The composition may be in the form of a lotion, serum, milk, O/W or W/O cream, gel, salve, ointment, powder, balm, patch, impregnated pad, soap, bar or foam.

In particular, the compositions for application to the scalp or the hair may be in the form of a hair care lotion, for example for daily or twice-weekly application, a shampoo or a hair conditioner, in particular for weekly or twice-weekly application, a liquid or solid soap for cleansing the scalp, for daily application, a product for shaping the hairstyle (lacquer, hair setting product, hairstyling gel), a treatment mask, or a foaming gel or cream for cleansing the hair. Same may also be in the form of a hair dye or hair mascara to be applied with a brush or with a comb.

Moreover, for application to the eyelashes or body hairs, the compositions of the invention may be in the form of a pigmented or nonpigmented mascara to be applied, with a brush, to the eyelashes or else to beard hair or moustache hair.

According to one particular embodiment, the composition according to the invention are in the form of a hair cream or lotion, a shampoo or hair conditioner, a hair mascara or an eyelash mascara.

The amounts of the various constituents of the physiological medium of the compositions according to the invention are those generally used in the fields under consideration. In addition, these compositions are prepared according to the usual methods.

When the composition is an emulsion, the proportion of the fatty phase may range from 1% to 80% by weight, and preferably from 1% to 50% by weight, relative to the total weight of the composition. The aqueous phase is adjusted according to the content of fatty phase and of compound(s) (A) and also to that of the possible additional ingredients, so as to obtain 100% by weight. In practice, the aqueous phase is from 5% to 99.9% by weight.

The fatty phase may contain fatty or oily compounds, which are liquid at ambient temperature (25° C.) and atmospheric pressure (760 mmHg), generally known as oils. These oils may or may not be compatible with one another and may form a macroscopically homogeneous liquid fatty phase or a two-phase or three-phase system.

The fatty phase may, in addition to the oils, contain waxes, gums, lipophilic polymers or “pasty” or viscous products containing solid parts and liquid parts.

The aqueous phase contains water and, optionally, an ingredient which is water-miscible in any proportion, for instance C₁ to C₈ lower alcohols such as ethanol or isopropanol, polyols such as propylene glycol, glycerol or sorbitol, or else acetone or ether.

The emulsifiers and coemulsifiers employed for obtaining a composition in emulsion form are those generally used in the cosmetics and pharmaceutical fields. The nature thereof also depends on the direction of the emulsion. In practice, the emulsifier and, optionally, the co-emulsifier are present, in the composition, in a proportion ranging from 0.1% to 30% by weight, preferably from 0.2% to 20% by weight, and better still from 0.2% to 8%. The emulsion may also contain lipid vesicles, and in particular liposomes.

When the composition is in the form of an oily solution or gel, the fatty phase may represent more than 90% of the total weight of the composition.

Advantageously, the composition is an aqueous, alcoholic or aqueous-alcoholic solution or suspension, and more preferably a water/ethanol solution or suspension. The alcoholic fraction may represent from 5% to 99.9%, and preferably from 8% to 80%.

For an application of mascara type, the composition is a wax-in-water or wax-in-oil dispersion, a gelled oil, or an aqueous gel, which may be pigmented or nonpigmented.

Additional Elements of the Invention:

The compositions of the invention may also comprise other cosmetic additives customarily used in the fields concerned, selected from among solvents, aqueous-phase or oily-phase thickeners or gelling agents, dyestuffs that are soluble in the medium of the composition, solid particles of the filler or pigment type, antioxidants, preservatives, fragrances, electrolytes, neutralizing agents, UV blockers such as sunscreens, film-forming polymers, cosmetic and pharmaceutical active agents with a beneficial action on the hair and the scalp, antidandruff agents, nutritive active agents, agents for combating grease, and calmatives, and mixtures thereof.

These additives may be present in the compositions according to the amounts generally used in the cosmetics and dermatological field, and in particular in a proportion of from 0.01% to 50% of the total weight of the composition, and preferably from 0.1% to 20% and, for example, from 0.1% to 10%. Depending on their nature, these adjuvants may be introduced into the fatty phase, into the aqueous phase and/or into lipid vesicles, and especially liposomes.

Of course, one skilled in this art will take care to select the optional additional additives and/or the amount thereof in such a way that the advantageous properties of the combination according to the invention are not, or are not substantially, impaired by the addition envisaged.

As solvents that may be used in the invention, exemplary are C₂ to C₈ lower alcohols or polyols, such as ethanol, isopropanol, propylene glycol, glycerol, polyethylene glycols, polyol ethers, such as dipropylene glycol monomethyl ether, and certain light cosmetic oils such as C₆ to C₁₆ alkanes.

As oils that can be used in the invention, exemplary are oils of mineral origin (liquid petroleum jelly, hydrogenated isoparaffin), oils of plant origin (liquid fraction of shea butter, sunflower oil, apricot oil, soya oil, avocado oil, alcohol or fatty acid), oils of animal origin (perhydrosqualene), synthetic oils (fatty acid esters, purcellin oil), silicone oils (linear or cyclic polydimethylsiloxanes, phenyl trimethicones) and fluoro oils (perfluoropolyethers). As waxes, exemplary are silicone waxes, beeswax, candelilla wax, rice wax, carnauba wax, jojoba wax, paraffin wax or polyethylene wax.

As emulsifiers that can be used in the invention, exemplary are glyceryl stearate or laurate, sorbitol stearates or oleates, alkyl dimethicone copolyols (with alkyl ≧8) and mixtures thereof for a W/O emulsion. Also exemplary are polyethylene glycol monostearate, isostearate or monolaurate, polyoxyethylenated sorbitol stearate or oleate, dimethicone copolyols and mixtures thereof for an O/W emulsion.

Anionic, nonionic, cationic or amphoteric surfactants may also be formulated. Among the anionic surfactants exemplary are alkyl sulfates, alkyl ether sulfates and alkyl ether carboxylates. Among the nonionic surfactants, most particularly exemplary are alkylpolyglucosides, polysorbates, oxyalkylenated or glycerolated fatty alcohols, and oxyalkylenated triglycerides. Among the amphoteric surfactants, particularly exemplary are betaines and camphodiacetates. Among the cationic surfactants, most particularly exemplary are fatty-chain alkyltrimethylammonium salts and diester quats.

As hydrophilic gelling agents according to the invention, exemplary are carboxyvinyl polymers (carbomer), acrylic copolymers such as acrylate/alkyl acrylate copolymers, polyacrylamides, polysaccharides such as hydroxyethyl- or hydroxypropylcelluloses, natural gums and clays, and as lipophilic gelling agents, exemplary are modified clays such as bentones, metal salts of fatty acids, such as aluminum stearates, hydrophobically treated silica, ethylcellulose and mixtures thereof.

The compositions may contain an additional active agent other than those mentioned above, which may be hydrophilic and selected from among proteins or protein hydrolysates, amino acids, urea, allantoin, sugars and sugar derivatives, water-soluble vitamins, plant extracts (those of Iridaceae plants or of soybean) and hydroxy acids (fruit acid, salicylic acid); or lipophilic and selected from retinol (vitamin A) and its derivatives, in particular ester (retinyl palmitate), tocopherol (vitamin E) and its derivatives, in particular ester (tocopheryl acetate or palmitate), vitamin F and its esters, and in particular esters of vitamin F and of glucose, essential fatty acids such as linoleic acid, eicosatetraenoic acid, linolenic acid and eicosatrienoic acid, or their esters and amides; ceramides; essential oils; salicylic acid and its derivatives, such as 5-n-octanoylsalicylic acid; esters of hydroxy acids; phospholipids, such as lecithin; anti-seborrhoeic agents; anti-virals; anti-pruritic agents; carotenoids such as β-carotene; lactones and corresponding salts thereof; phenols and polyphenols such as flavonoids; and mixtures thereof.

The anti-androgenic agents that can be used include in particular steroidal or non-steroidal inhibitors of 5α-reductase, such as finasteride and the compounds described in U.S. Pat. No. 5,516,779, cyprosterone acetate, azelaic acid, its salts and its derivatives and the compounds described in U.S. Pat. No. 5,480,913, flutamide, the compounds described in U.S. Pat. Nos. 5,411,981, 5,565,467 and 4,910,226, and also oxendolone, spironolactone, diethylstilbestrol and flutamide.

The antimicrobial or antifungal compounds may be selected from among selenium derivatives, ketoconazole, octopirox, triclocarban, triclosan, zinc pyrithione, itraconazole, metronidazole, crotamiton or pyrethrinoids, Asiatic acid, hinokitiol, mipirocine, tetracyclines, in particular erythromycin and the compounds described in EP0680745, clinycine hydrochloride, benzoyl or benzyl peroxide and minocycline and compounds belonging to the imidazole group, such as econazole, ketoconazole or miconazole or salts thereof, nicotinic acid esters, including in particular tocopherol nicotinate, benzyl nicotinate and C₁-C₆ alkyl nicotinates, for instance methyl nicotinate or hexyl nicotinate.

The anti-inflammatories may be selected from among steroidal anti-inflammatories, such as glucocorticoids, corticosteroids (for example: hydrocortisone) and non-steroidal anti-inflammatories, such as glycyrrhetinic acid, α-bisabolol, benzydamine and the compounds described in EP0770399, WO 94/06434 and FR2268523. The retinoids may be selected from retinoic acid, isotretinoin, acitretin and tazarotene.

As other additional compounds that are active agents for promoting hair growth and/or limiting hair loss that can be included in the compositions according to the invention, exemplary are 2,4-diaminopyrimidine-3-N-oxide (or aminexil), 6-O-[(9Z,12Z)-octadeca-9,12-dienoyl]hexapyranose, benzalkonium chloride, benzethonium chloride, phenol, oestradiol, chlorpheniramine maleate, chlorophylline derivatives, cholesterol, cysteine, methionine, menthol, peppermint oil, calcium pantothenate, panthenol, resorcinol, protein kinase C activators, glycosidase inhibitors, glycosaminoglycanase inhibitors, pyroglutamic acid esters, hexosaccharidic or acylhexosaccharic acids, substituted aryl ethylenes, N-acylamino acids, flavonoids, ascomycin derivatives and analogues, histamine antagonists, saponins, proteoglycanase inhibitors, oestrogen agonists and antagonists, pseudoterines, cytokines and growth factor promoters, IL-1 or IL-6 inhibitors, IL-10 promoters, TNF inhibiters, benzophenones and hydantoin; vitamins such as vitamin D, vitamin B12 analogues and pantothenol; triterpenes, for instance ursolic acid and the compounds described in U.S. Pat. Nos. 5,529,769, 5,468,888 and 5,631,282; antipruritic agents, for instance thenaldine, trimeprazine or cyproheptadine; hormones such as oestriol or analogues thereof, thyroxine and salts thereof, progesterone; FP receptor (prostaglandin type F receptor) agonists such as latanoprost, bimatoprost, travoprost or unoprostone; and mixtures thereof.

The compositions for topical application may be encapsulated, and in particular in liposomal form, as described in particular in WO94/22468. Thus, the compound encapsulated in the liposomes can be delivered selectively at the hair follicle.

The compositions according to the invention may be applied to the alopecic areas of the scalp and of the hair of an individual, and optionally left in contact for several hours and optionally rinsed off.

It is possible, for example, to apply the composition in the evening, to keep said composition in contact overnight and, optionally, to shampoo in the morning. These applications may be repeated daily for one or more months depending on individuals.

Advantageously, in the regime or regimen according to the invention, from 5 to 500 μl of a solution or composition as defined above are applied to the areas of the scalp that are to be treated.

In order to further illustrate the present invention and the advantages thereof, the following specific examples are given, it being understood that same are intended only as illustrative and in nowise limitative. In said examples to follow, all parts and percentages are given by weight, unless otherwise indicated.

### Examples

### Example 1 of a Lotion

| Ingredients | Amount (% by weight) |
|---|---|
| L-arginine | 1.5 |
| MADECASSOSIDE marketed | 0.3 |
| by Laboratoires Roche Nicholas | |
| PEG-40 Hydrogenated Castor Oil | 0.3 |
| Denatured 96° ethyl alcohol | 21.9 |
| Deionized water | qs 100 |

### Example 2 of an Emulsion

| Ingredients | Amount (% by weight) |
|---|---|
| L-arginine | 1.5 |
| PEG-8 isostearate | 0.25 |
| Behenyltrimonium chloride | 0.25 |
| Protected avocado oil | 0.65 |
| MADECASSOSIDE | 0.3 |
| Liquid jojoba wax | 0.65 |
| Cyclopentadimethylsiloxane | 0.45 |
| Polydimethyl/methylaminoethylaminopro- | 0.75 |
| pylsiloxane in a nonionic emulsion, | |
| protected | |
| Glycerol | 0.62 |
| Denatured 96° ethyl alcohol | 14.3 |
| Fragrance | 0.4 |
| 2-Amino-2-methyl-1-propanol | Qs pH 7 |
| Deionized water | qs 100 |

Each patent, patent application, publication, text and literature article/report cited or indicated herein is hereby expressly incorporated by reference in its entirety.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

What is claimed is:

1. A care composition for human keratin fibers, comprising, formulated into a physiologically acceptable medium, at least:
a) one or more compounds selected from among arginines and/or the organic or inorganic acid salts and/or derivatives thereof;
b) a compound or a mixture of compounds of formula (I) below: 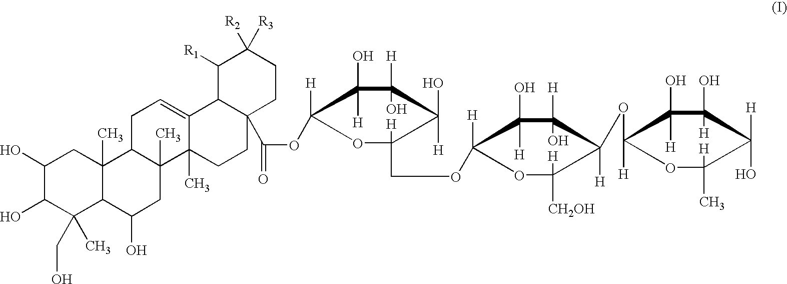
in which:
R₁═H or —CH₃; R₂═H or —CH₃; R₃═—CH₃, with the proviso that R₁ and R₂ are not simultaneously H, or else an extract of Centella asiatica comprising said compound or mixture of compounds of formula (I).

2. The care composition as defined by claim 1, wherein the compound(s) of formula (I) is (are) selected from madecassoside and/or terminoloside.

3. The care composition as defined by claim 2, comprising a mixture of madecassoside and of terminoloside.

4. The care composition as defined by claim 3, wherein the mixture of madecassoside and of terminoloside has a madecassoside content ranging from 30% to 70% by weight relative to the total weight of the mixture.

5. The care composition as defined by claim 3, comprising an extract of Centella asiatica comprising more than 95% by weight of the madecassoside/terminoloside mixture, relative to the weight of the extract.

6. The care composition as defined by claim 1, wherein the compound or the mixture of compounds of formula (I) is present in an amount ranging from 0.001% to 20% by weight, relative to the total weight of the composition.

7. The care composition as defined by claim 1, comprising an arginine selected from among D-arginine, D,L-arginine and L-arginine.

8. The care composition as defined by claim 1, comprising an arginine salt selected from among hydrochlorides, glutamates, butyrates and glycolates.

9. The care composition as defined by claim 1, comprising an L-arginine derivative selected from among the C₁-C₄ alkyl esters of L-arginine and the oligomers of L-arginine comprising from 7 to 15 L-arginine units.

10. The care composition as defined by claim 1, comprising L-arginine.

11. The care composition as defined by claim 1, wherein the arginine(s) and/or the salts and/or derivatives thereof are present in an amount ranging from 0.01% to 5% of the total weight of the composition.

12. The care composition as defined by claim 1, comprising at least one cosmetic additive selected from among solvents, aqueous-phase or oily-phase thickeners or gelling agents, dyestuffs that are soluble in the medium of the composition, solid particles of the filler or pigment type, antioxidants, preservatives, fragrances, electrolytes, neutralizing agents, UV blockers, film-forming polymers, cosmetic and pharmaceutical active agents having a beneficial action on the hair and the scalp, antidandruff agents, nutritive active agents, agents for combating grease, and calmatives, and mixtures thereof.

13. The care composition as defined by claim 1, further comprising at least one additional compound for promoting the stimulation and/or induction of hair growth and/or limiting the loss of human keratin fibers.

14. A regime or regimen for inducing and/or stimulating the growth of human keratin fibers and/or preventing the loss and/or increasing the density thereof, comprising administering to an individual in need of such treatment, a thus effective amount of a combination of one or more compounds selected from among arginines and/or the organic or inorganic acid salts and/or derivatives thereof and of a compound or of a mixture of compounds of formula (I) as defined by claim 1 or else of an extract of Centella asiatica comprising the compound or the mixture of compounds of formula (I).

15. A cosmetic treatment regime or regimen for human keratin fibers and/or the scalp, for stimulating and/or inducing the growth of said fibers, and/or preventing the loss and/or increasing the density thereof, comprising applying to said fibers and/or the scalp, a cosmetic care composition as defined by claim 1, leaving said composition in contact with these keratin fibers and/or with the scalp, and optionally rinsing these keratin fibers and/or the scalp.
